(19) United States
(12) Patent Application Publication
(10) Pub. No.: US 2026/0155206 A1
(43) Pub. Date: Jun. 4, 2026

(54) PATIENT SELECTION BY PREDICTING TARGET GENE ESSENTIALITY USING MACHINE LEARNING

(71) Applicant: Genzyme Corporation, Cambridge, MA (US)

(72) Inventor: Mi Yang, Cambridge, MA (US)

(21) Appl. No.: 19/177,332

(22) Filed: Apr. 11, 2025

Related U.S. Application Data
(60) Provisional application No. 63/634,863, filed on Apr. 16, 2024.

(51) Int. Cl. G16B 25/10; G16B 40/20; G16H 20/10

(52) U.S. Cl. CPC G16B 25/10; G16B 40/20; G16H 20/10

(57) ABSTRACT

Methods, systems, and apparatus, including computer programs encoded on a computer storage medium, for determining whether to select a patient, e.g., to receive a drug that targets a target gene. In one aspect, a method comprises: obtaining gene expression data for a collection of cells from a patient; processing a model input comprising the gene expression data using a machine learning model, in accordance with values of a set of machine learning model parameters, to generate a predicted gene essentiality score for the target gene; and determining whether to select the patient based at least in part on the predicted gene essentiality score for the target gene.

### CROSS REFERENCE TO RELATED APPLICATION

The present application claims priority to U.S. Provisional Application No. 63/634,863, filed on Apr. 16, 2024, the disclosures of which is incorporated herein by reference.

### BACKGROUND

This specification relates to processing data using machine learning models.

Machine learning models receive an input and generate an output, e.g., a predicted output, based on the received input. Some machine learning models are parametric models and generate the output based on the received input and on values of the parameters of the model.

A gene may be referred to as an “essential” gene for cells in a collection of cells, e.g., if the gene is essential for viability of the cells in the collection of cells, e.g., such that the loss of the function of the gene in cells in the collection of cells severely impairs or prevents critical biological processes of the cells.

### SUMMARY

This specification describes a system implemented as computer programs on one or more computers in one or more locations that can process gene expression data for a collection of genes from a patient, e.g., obtained by performing a biopsy of a cancerous tumor of the patient, to generate a predicted gene essentiality score for a target gene. The system can determine treatment recommendations, such as whether the patient should receive a drug that targets the target gene, based on the predicted gene essentiality score for the target gene.

According to one aspect, there is provided a method performed by one or more computers, the method comprising: obtaining gene expression data for a collection of cells from a patient; processing a model input comprising the gene expression data using a machine learning model, in accordance with values of a set of machine learning model parameters, to generate a predicted gene essentiality score for a target gene; and providing the gene essentiality score for the target gene.

In some implementations, the predicted gene essentiality score for the target gene characterizes whether the target gene is essential for viability of cells in the collection of cells from the patient.

In some implementations, the gene expression data comprises bulk gene expression data that defines, for each gene in a set of genes, an expression level of the gene across the collection of cells from the patient.

In some implementations, the gene expression data comprises cell type-specific gene expression data for each of one or more target cell types; and wherein for each target cell type, the cell type-specific gene expression data for the target cell type defines, for each gene in a set of genes, a predicted expression level of the gene across cells of the target cell type in the collection of cells from the patient.

In some implementations, the method further comprises generating the cell type-specific gene expression data by operations including: obtaining bulk gene expression data that defines, for each gene in the set of genes, an expression level of the gene across the collection of cells from the patient; processing the bulk gene expression data to generate cell type-specific gene expression data for each cell type in a set of possible cell types, wherein each of the one or more target cell types are included in the set of possible cell types; and extracting the cell type-specific gene expression data for each of the one or more target cell types from the cell type-specific gene expression data for the set of possible cell types.

In some implementations, wherein the one or more target cell types include epithelial cells.

In some implementations, the one or more target cell types include exactly one target cell type.

In some implementations, the set of possible cell types include one or more of: epithelial cells, pericyte cells, natural killer cells, macrophage cells, fibroblast cells, regulatory T cells, conventional dendritic cells, CD8-positive alpha-beta T cells, B cells, endothelial cells, CD4-positive alpha-beta T cells, effector CD8-positive alpha-beta T cells, or unassigned cells.

In some implementations, wherein the gene expression data included in the model input to the machine learning model defines a respective expression level of each gene in a set of genes.

In some implementations, the set of genes includes only genes that participate in a biochemical pathway associated with the target gene.

In some implementations, the particular disease is a type of cancer.

In some implementations, the machine learning has been trained on a set of training examples by a machine learning training technique.

In some implementations, each training example in the set of training examples includes: (i) a training input to the machine learning model that comprises gene expression data for a training collection of cells, and (ii) a target gene essentiality score for the target gene.

In some implementations, training the machine learning model on the set of training examples comprises, for each training example: training the machine learning model to optimize an objective function that measures a discrepancy between: (i) the target gene essentiality score specified by the training example, and (ii) a predicted gene essentiality score generated by processing the training input of the training example using the machine learning model.

In some implementations, for one or more of the training examples, the training example is derived from a cell line, and the training input of the training example comprises gene expression data for cells in the cell line.

In some implementations, the machine learning model comprises one or more of: a neural network model, or a support vector machine learning, or a random forest model, or a linear regression model.

In some implementations, the collection of cells from the patient are obtained from a biopsy of the patient.

In some implementations, the biopsy comprises one or more of: a needle biopsy, or a punch biopsy, or an incisional biopsy, or an excisional biopsy, or an endoscopic biopsy, or a bone marrow biopsy, or a blood biopsy.

In some implementations, the collection of cells are obtained from a biopsy of a cancerous tumor of the patient.

In some implementations, the cancerous tumor of the patient is a kidney cancer tumor, or a bone cancer tumor, or a lung cancer tumor, or a bladder cancer tumor, or an ovarian cancer tumor, or a colon colorectal cancer tumor, or an endometrial uterine cancer tumor, or a breast cancer tumor, of a pancreatic cancer tumor, or a lymphoma cancer tumor, or a brain cancer tumor, or a bile duct cancer tumor, or a neuroblastoma cancer tumor, or a sarcoma cancer tumor, or a skin cancer tumor, or a gastric cancer tumor, or a head and neck cancer tumor, or an esophageal cancer tumor.

In some implementations, providing the predicted gene essentiality score for the target gene comprises: determining whether the patient should receive a drug that target the target gene based at least in part on the predicted gene essentiality score.

In some implementations, determining whether the patient should receive a drug that target that target gene based at least in part on the predicted gene essentiality score comprises:
determining whether the patient should receive the drug that targets the target gene based at least in part on whether the predicted gene essentiality score satisfies a threshold.

In some implementations, the method further comprises, in response to determining that the patient should receive the drug, causing the drug to be administered to the patient.

In some implementations, providing the predicted gene essentiality score for the target gene comprises: determining a likelihood that the patient will respond to a drug that targets the target gene based at least in part on the predicted gene essentiality score.

According to another aspect, there is provided a system comprising: one or more computers; and one or more storage devices communicatively coupled to the one or more computers, wherein the one or more storage devices store instructions that, when executed by the one or more computers, cause the one or more computers to perform operations of the methods described herein.

According to another aspect, there are provided one or more non-transitory computer storage media storing instructions that when executed by one or more computers cause the one or more computers to perform operations of the methods described herein.

Particular embodiments of the subject matter described in this specification can be implemented so as to realize one or more of the following advantages.

This specification describes a prediction system that can process gene expression data for a collection of cells from a patient, e.g., obtained by performing a biopsy of a cancerous tumor of the patient, to determine, e.g., whether the patient should receive a drug that targets a particular target gene. The prediction system thus enables applications in precision medicine, where each patient is evaluated individually based on their patient-specific genetic makeup to determine whether the patient would benefit from receiving the drug, which can provide improved treatment efficacy for patients. The prediction system therefore provides a technical improvement in the field of precision medicine.

To determine whether a patient should receive a drug that targets a target gene, the system can process gene expression data for the patient using a machine learning model to generate a predicted gene essentiality score that characterizes whether the target gene is an “essential gene” for the collection of cells from patient. (As essential gene may refer to a gene that is essential for cell viability, as will be described in more detail throughout this specification). The system can then determine that the patient should receive the drug targeting the target gene, e.g., if the predicted gene essentiality score satisfies (e.g., exceeds) a threshold. Intuitively, for example, the more essential the target gene is for viability of cancerous cells in the patient, the higher the likelihood that the drug targeting the target gene will be effective for treating the patient.

The prediction system can generate treatment recommendations, e.g., for whether a patient should receive a drug, using significantly fewer computational resources (e.g., memory and computing power) than are required for some conventional approaches. More specifically, generating accurate patient-specific treatment recommendations is a difficult prediction task, and to account for this, some conventional systems leverage machine learning models that have complex architectures and that process high-dimensional, multi-modal patient data. In contrast, the prediction system described in this specification performs the easier task of predicting gene essentiality, and then exploits the relationship between gene essentiality and drug effectiveness to generate a treatment recommendation, e.g., by comparing the gene essentiality score to a threshold. The prediction system can therefore generate treatment recommendations using a less complex machine learning model that uses fewer computational resources (e.g., by performing fewer arithmetic operations) than conventional approaches. The post-processing step of translating predicted gene essentiality to a treatment recommendation can be implemented using operations such as thresholding that only negligibly increase the computational cost of the prediction system. The prediction system therefore provides an improvement in the functioning of a computer, in particular, by reducing consumption of computational resources.

Prior to processing gene expression data using the machine learning model to generate a predicted gene essentiality score, the prediction system can pre-process the gene expression data. For instance, the prediction system can transform bulk gene expression data (that characterizes gene expression over all the cells in a collection of cells obtained from a patient) to cell type-specific gene expression data (that, for each cell type in a collection of cell types, characterizes gene expression data over only cells of the cell type in the collection of cells). The prediction system can extract cell type-specific gene expression data for one or more “target” cell types, and provide the target cell type-specific gene expression data for processing by the machine learning model. The prediction system can select the one or more target cell types, e.g., as cell types from which tumor cells of a cancer of interest are frequently derived (e.g., epithelial cells), so that the target cell type-specific gene expression data characterizes the tumor more directly than the bulk gene expression data which characterizes both the tumor and the tumor microenvironment. Pre-processing the gene expression data can increase the accuracy of the machine learning model and, by extension, increase the precision and quality of treatment recommendations generated based on the output of the machine learning model.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example prediction system.

FIG. 2 is a flow diagram of an example process for generating a prediction characterizing a patient based on a predicted gene essentiality score for a target gene in a collection of cells obtained by performing a biopsy on the patient.

FIG. 3 is a flow diagram of an example process for pre-processing bulk gene expression data for a collection of cells, e.g., prior to the gene expression data being processed by a machine learning model to generate a predicted gene essentiality score for a target gene.

FIG. 4 is a flow diagram of an example process for training a machine learning model to generate a predicted gene essentiality score for a target gene.

FIG. 5 shows a plot that illustrates an example of the effects of transforming the bulk gene expression data to cell type-specific gene expression data prior to processing the gene expression data using the machine learning model to predict the gene essentiality score for the target gene.

FIG. 6 shows a plot that illustrates, for each cell type in a set of cell types (as shown on the horizontal axis of the plot), a Spearman's correlation (as shown on the vertical axis of the plot) between: (i) predicted cell type-specific gene expression levels for the cell type, and (ii) ground truth cell type-specific gene expression levels for the cell type.

Like reference numbers and designations in the various drawings indicate like elements.

### DETAILED DESCRIPTION

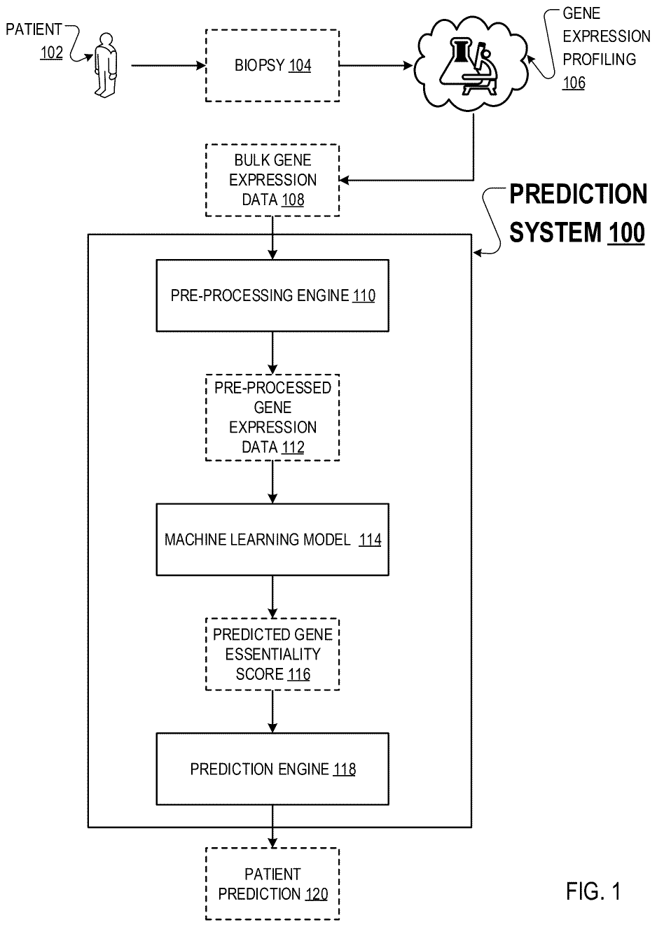

FIG. 1 shows an example prediction system 100. The prediction system 100 is an example of a system implemented as computer programs on one or more computers in one or more locations in which the systems, components, and techniques described below are implemented.

The prediction system is configured to receive bulk gene expression data 108 generated by performing gene expression profiling 106 on a collection of cells obtained by performing a biopsy 104 on a patient 102. The prediction system 100 processes the bulk gene expression data 108 to generate a prediction 120 characterizing the patient 102, as will be described in more detail below.

The collection of cells obtained by performing the biopsy 104 on the patient 102 can include any appropriate number of cells, e.g., at least 1000 cells, or at least 100,000 cells, or at least 1,000,000 cells.

The collection of cells can be obtained from the patient 102 using any appropriate biopsy technique, e.g., a needle biopsy, or punch biopsy, or incisional biopsy, or excisional biopsy, or endoscopic biopsy, or bone marrow biopsy, or blood biopsy.

The patient 102 may have a disease, e.g., cancer, and the collection of cells obtained by performing the biopsy 104 on the patient 102 may include cancerous cells. For instance, the collection of cells may be obtained by performing a biopsy on a cancerous tumor of the patient, e.g., a kidney cancer tumor, or a bone cancer tumor, or a lung cancer tumor, or a bladder cancer tumor, or an ovarian cancer tumor, or a colon colorectal cancer tumor, or an endometrial uterine cancer tumor, or a breast cancer tumor, of a pancreatic cancer tumor, or a lymphoma cancer tumor, or a brain cancer tumor, or a bile duct cancer tumor, or a neuroblastoma cancer tumor, or a sarcoma cancer tumor, or a skin cancer tumor, or a gastric cancer tumor, or a head and neck cancer tumor, or an esophageal cancer tumor.

The bulk gene expression data 108 can define, for each gene in a set of genes, an expression level of the gene across the collection of cells from the patient 102. The bulk gene expression data 108 can be generated by applying any appropriate gene expression profiling technique 106 to the collection of cells from the patient, e.g., bulk ribonucleic acid sequencing (RNA-seq), or microarray sequencing, or quantitative real-time polymerase chain reaction (qRT-PCR) sequencing, and so forth.

The bulk gene expression data 108 can define respective expression levels for any appropriate number of genes, e.g., at least 1000 genes, or at least 10,000 genes, or at least 40,000 genes. In some cases, the bulk gene expression data 108 may define gene expression levels for a substantial portion of the genome, while in other cases, the bulk gene expression data 108 may define gene expression data for a much smaller set of genes, e.g., a set of genes that specifically participate in a particular biochemical pathway, e.g., a biochemical pathway associated with a target gene.

The prediction system 100 can process the bulk gene expression data 108 to generate any appropriate prediction 120 characterizing the patient 102. A few examples of possible patient predictions 120 that can be generated by the prediction system 100 are described next.

In one example, the prediction system 100 can generate a patient prediction 120 that defines a prediction for whether the patient 102 should receive a particular drug, e.g., to treat a disease affecting the patient 102.

In another example, the prediction system 100 can generate a patient prediction 120 that defines a likelihood that a disease affecting the patient 102 will respond to a drug if the drug is administered to the patient 102. (A disease can be said to “respond” to a drug, e.g., if a metric characterizing the symptoms, severity, or progression of the disease changes-e.g., improves-by at least a threshold amount or proportion).

The prediction system 100 can provide the patient prediction 120, e.g., by storing the patient prediction 120 in a memory, or by presenting the patient prediction 120 on a display of a user device, or by transmitting the patient prediction 120 across a data communication network, or any combination thereof.

The prediction system 100 can be implemented in a clinical workflow in a healthcare environment, e.g., a hospital. For instance, a healthcare provider may obtain a biopsy 104 from a patient 102 having a disease, provide the biopsy 104 for gene expression profiling 106, and then provide the resulting bulk gene expression data 108 for processing by the prediction system 100. The prediction system 100 can process the bulk gene expression data 108 to generate a patient prediction 120, and then store the patient prediction in an electronic medical record (EMR) associated with the patient 102. The healthcare provider can access the EMR associated with the patient, e.g., by way of an application running on a user device of the healthcare provider, e.g., a smartphone, tablet, or personal computer. In implementations where the patient prediction 120 defines a prediction for whether the patient should receive a drug or whether the patient will respond to a drug, the healthcare provider can determine whether to administer the drug to the patient based at least in part on the patient prediction 120.

The prediction system 100 can be used to define inclusion or exclusion criteria for a clinical trial for a drug. (A “clinical trial” for a drug can refer to a research study conducted to evaluate the safety and effectiveness of the drug). For instance, the inclusion criteria for a clinical trial may be defined to require that, in order for a patient to be eligible for inclusion in the clinical trial for a drug, the prediction system 100 must predict that the patient should receive the drug or is likely to respond to the drug. As another example, the exclusion criteria for a clinical trial may be defined such that a patient is excluded from the clinical trial if the prediction system 100 predicts that the patient should not receive the drug or is not likely to respond to the drug.

The prediction system can include: (i) a pre-processing engine 110, (ii) a machine learning model 114, and (iii) a prediction engine 118, which are each described in more detail next (and throughout this specification).

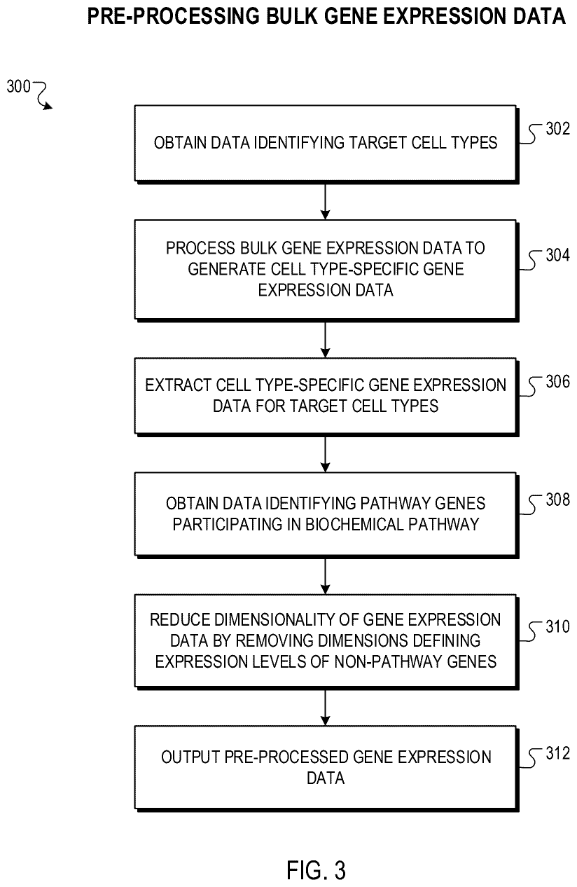

The pre-processing engine 110 is configured to process the bulk gene expression data 108 to generate pre-processed gene expression data 112. The pre-processing engine 110 can pre-process the bulk gene expression data 108 in any of a variety of possible ways. For instance, the pre-processing engine 110 can transform the bulk gene expression data to generate cell type-specific gene expression data for one or more target cell types. In more detail, the bulk gene expression data defines gene expression levels over the entire collection of cells from the patient, and in particular, over all the cell types that are included in the collection of cells from the patient. In contrast, the cell type-specific gene expression data for a target cell type defines, for each gene in a set of genes, a predicted expression level of the gene across cells of the target cell type in the collection of cells from the patient. An example process for pre-processing the bulk gene expression data 108 is described in more detail with reference to FIG. 3.

The machine learning model 114 is configured to process the pre-processed gene expression data 112, in accordance with values of a set of machine learning model parameters, to generate a predicted gene essentiality score 116 for a target gene. The predicted gene essentiality score 116 can characterize whether the target gene is an essential gene for cells in the collection of cells, e.g., in that the target gene is essential for viability of the cells in the collection of cells, e.g., such that the loss of the function of the gene in cells in the collection of cells severely impairs or prevents critical biological processes of the cells.

The target gene can be any appropriate gene in the human genome. In particular, the target gene can be a gene that is targeted by a drug of interest. A drug can be referred to as “targeting” a gene, e.g., if the drug interacts with and influences the function of the gene or a protein product that the gene encodes. For instance, a drug can target a gene by enhancing or inhibiting the production of messenger ribonucleic acid (mRNA) for the gene, thereby increasing or decreasing the production of a protein coded for by the gene. As another example, a drug can target a gene by binding to a protein produced by the gene in order to inhibit or activate the protein, or to change the stability of the protein, or to alter the interaction of the protein with other molecules.

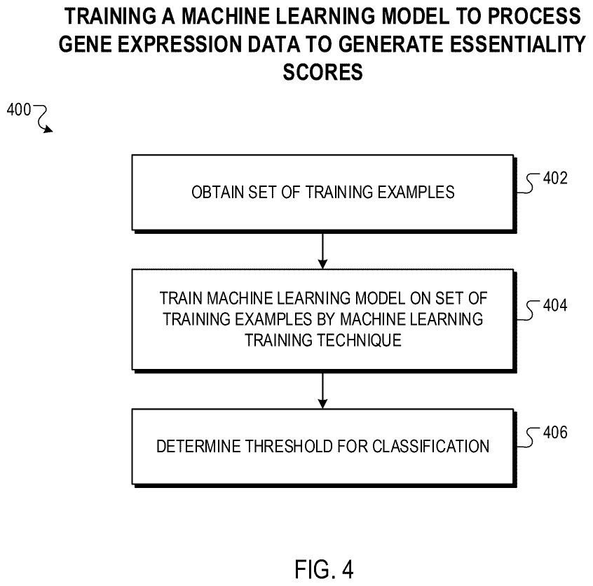

The prediction system 100 can train the machine learning model on a set of training examples by a machine learning training technique. An example process for training the machine learning model is described in detail below with reference to FIG. 4.

In some implementations, the pre-processing engine 110 is excluded from the prediction system 100, and the machine learning model 114 directly processes the bulk gene expression data 108 rather than processing the pre-processed gene expression data generated by the pre-processing engine 110.

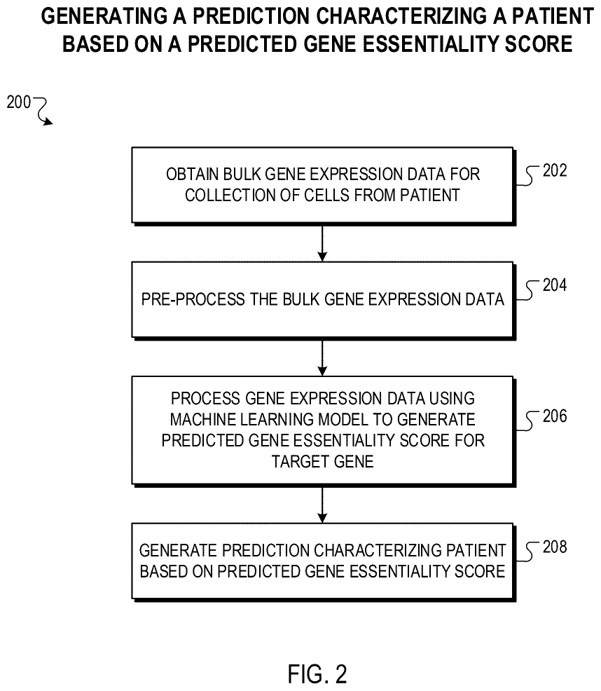

The prediction engine 118 is configured to process the predicted gene essentiality score 116 to generate the patient prediction 120. For instance, in implementations where the predicted gene essentiality score 116 defines a likelihood that the target gene is an essential gene, the prediction engine 118 can determine whether the predicted gene essentiality score satisfies (e.g., exceeds) a predefined threshold. In response to determining that the predicted gene essentiality score satisfies the threshold, the prediction engine 118 can generate a first patient prediction 120, e.g., predicting that the patient 102 should receive a drug targeting the target gene, or predicting that the patient will respond to a drug targeting the target gene. In response to determining that the predicted gene essentiality score does not satisfy the threshold, the prediction engine 118 can generate a second (different) patient prediction 120, e.g., predicting that the patient should not receive a drug targeting the target gene, or predicting that the patient will not respond to a drug targeting the target gene. Example techniques for processing the predicted gene essentiality score to generate the patient prediction are described in more detail below with reference to FIG. 2.

FIG. 2 is a flow diagram of an example process 200 for generating a prediction characterizing a patient based on a predicted gene essentiality score for a target gene in a collection of cells obtained by performing a biopsy on the patient. For convenience, the process 200 will be described as being performed by a system of one or more computers located in one or more locations. For example, a prediction system, e.g., the prediction system 100 of FIG. 1, appropriately programmed in accordance with this specification, can perform the process 200.

The system obtains bulk gene expression data for a collection of cells from a patient (202). The bulk gene expression data defines, for each gene in a set of genes, an expression level of the gene across the collection of cells from the patient. The collection of cells can be obtained from the patient, e.g., by way of a biopsy, e.g., on a cancerous tumor of the patient, and the bulk gene expression data can be generated by applying an appropriate gene expression profiling technique to the collection of cells from the patient.

The system pre-processes the bulk gene expression data (204). For instance, the system can pre-process the bulk gene expression data to generate cell type-specific gene expression data for each of one or more target cell types. An example process for pre-processing the bulk gene expression data is described in detail below with reference to FIG. 3.

The system processes a model input that includes the pre-processed gene expression data (e.g., as generated at step 204), using a machine learning model and in accordance with values of a set of machine learning model parameters of the machine learning model, to generate a predicted gene essentiality score for the target gene (206). The predicted gene essentiality score for the target gene may characterize whether the target gene is essential for viability of cells in the collection of cells from the patient.

In some cases, the predicted gene essentiality score is a scalar numerical value from a continuous range of possible gene essentiality scores (e.g., the interval [0,1]) which defines a likelihood that the target gene is an essential gene for cells in the collection of cells from the patient.

In some cases, the predicted gene essentiality score is a scalar numerical value from a continuous range of possible gene essentiality scores which defines a predicted degree of fitness reduction caused by down-regulating or knocking out the target gene. For instance, the predicted gene essentiality score can define a predicted amount of impairment of cell survival or proliferation that is caused by down-regulating or knocking out the target gene.

In some cases, the predicted gene essentiality is a binary score (e.g., that can assume binary values 0 or 1) that defines a classification of the collection of cells from the patient into: (i) an essential gene class, indicating that the target gene is essential for the collection of cells, and (ii) a non-essential gene class, indicating that the target gene is non-essential for the collection of cells.

The machine learning model can have any appropriate type of machine learning model architecture that enables the machine learning model to perform its described functions, e.g., processing gene expression data to generate a predicted gene essentiality score for the target gene. For instance, the machine learning model can include one or more of: a neural network, or a random forest, or a support vector machine, or a linear regression model.

Prior to using the machine learning model as part of the process 200, the system trains the machine learning on a set of training examples by a machine learning training technique. An example process for training the machine learning model is described in detail below with reference to FIG. 4.

Optionally, the process 200 can exclude the step 204 of pre-processing the bulk gene expression data, and the machine learning model can directly process the bulk gene expression data (at step 206) rather than processing the pre-processed gene expression data.

The system generates a prediction characterizing the patient based on the predicted gene essentiality score for the target gene (208).

In some implementations, the predicted gene essentiality score is a scalar numerical value from a continuous range of possible gene essentiality scores (as described above), and the system generates the prediction characterizing the patient by determining whether the predicted gene essentiality score satisfies (e.g., exceeds) a threshold. For instance, the system can generate a prediction that the patient should receive a drug targeting the target gene if the predicted gene essentiality score satisfies the target threshold, but not otherwise. As another example, the system can generate a prediction that the patient will respond to a drug targeting the target gene if the predicted gene essentiality score satisfies the threshold, but not otherwise.

The system can determine the threshold to which the predicted gene essentiality score is compared in any of a variety of possible ways. For the instance, the system can set the threshold to a value that is manually selected by a user of the system, e.g., by way of a user interface (e.g., a graphical user interface) or an application programming interface (API) made available by the system. As another example, the system can determine the threshold by an automated process during the training of the machine learning model. An example technique for determining the threshold as part of the training of the machine learning model is described below with reference to FIG. 4.

In some implementations, the predicted gene essentiality score directly classifies the target gene into: (i) an essential gene class, or (ii) a non-essential gene class (as described above), and the system generates the prediction characterizing the patient by mapping the classification of the target gene onto a corresponding prediction in accordance with a predefined prediction rule. For instance, in response to determining that the target gene is classified as an essential gene for the collection of cells, the system can generate a prediction that the patient should receive a drug targeting the target gene. In response to determining that the target gene is classified as a non-essential gene for the collection of cells, the system can generate a prediction that the patient should not receive the drug targeting the gene. As another example, in response to determining that the target gene is classified as an essential gene for the collection of cells, the system can generate a prediction that the patient will respond to a drug targeting the target gene. In response to determining that the target gene is classified as a non-essential gene for the collection of cells, the system can generate a prediction that the patient will not respond to the drug targeting the target gene.

In some implementations, the system processes a model input that includes the predicted gene essentiality score, using a clinical recommendation machine learning model and in accordance with values of a set of clinical recommendation machine learning model parameters, to generate the prediction characterizing the patient. The prediction characterizing the patient can, as described above, be a prediction for whether the patient should receive a drug targeting the target gene, or be a prediction for whether the patient will respond to a drug targeting the target gene. However, in contrast to implementations where the prediction is generated in accordance with a set of manually defined rules, the clinical recommendation machine learning model generates the prediction by operating on a model input that includes the predicted gene essentiality score by machine learned operations encoded in the values of the set of clinical recommendation machine learning model parameters.

The model input to the clinical recommendation machine learning can include other data in addition to the predicted gene essentiality score for the target gene, e.g., including one or more of: demographic information of the patient (e.g., age and gender), medical history of the patient (e.g., previous diagnoses, and previous treatments and responses), current health status of the patient (e.g., current symptoms, current medications, and vital signs), laboratory tests and biomarkers (e.g., data from blood tests, genetic tests, and medical imaging), and so forth.

The system can train the clinical recommendation machine learning model on a set of training examples by a machine learning training technique. Each training example can correspond to a training patient and can define: (i) a training input to the clinical recommendation machine learning model, and (ii) a target clinical recommendation. The target clinical recommendations can be generated, e.g., by manual annotation (where a human expert, e.g., a physician, manually defines the target clinical recommendation).

The clinical recommendation machine learning model can have any appropriate type of machine learning model architecture that enables the clinical recommendation machine learning model to perform its described functions, e.g., processing a model input that includes a predicted gene essentiality score to generate a predicted clinical recommendation. For instance, the clinical recommendation machine learning model can include one or more of: a neural network, or a random forest, or a support vector machine, or a linear regression model.

FIG. 3 is a flow diagram of an example process 300 for pre-processing bulk gene expression data for a collection of cells, e.g., prior to the gene expression data being processed by a machine learning model to generate a predicted gene essentiality score for a target gene. For convenience, the process 300 will be described as being performed by a system of one or more computers located in one or more locations. For example, a prediction system, e.g., the prediction system 100 of FIG. 1, appropriately programmed in accordance with this specification, can perform the process 300.

The system obtains data identifying one or more target cell types from a set of possible cell types (302). The set of possible cell types can include any appropriate number of possible cell types, e.g., at least 3 possible cell types, or at least 10 possible cell types, or at least 50 possible cell types. For example, the set of possible cell types can include one or more of: epithelial cells, pericyte cells, natural killer cells, macrophage cells, fibroblast cells, regulatory T cells, conventional dendritic cells, CD8-positive alpha-beta T cells, B cells, endothelial cells, CD4-positive alpha-beta T cells, effector CD8-positive alpha-beta T cells, or unassigned cells.

The number of possible cell types designated as target cell types is less than the total number of possible cell types, i.e., such that one or more of the possible cell types in the set of possible cell types are not designated as target cell types. In some cases, exactly one cell type in the set of possible cell types is designated as a target cell type.

The target cell types can be selected in any appropriate way. A few example criteria for selecting which cell types from the set of possible cell types to designate as target cell types are described next.

In one example, the target cell types can be selected as one or more cell types from which cancerous tumor cells of a cancer of interest are most frequently derived. For instance, for carcinomas such as breast cancer, prostate cancer, colorectal cancer, and lung cancer, cancerous tumor cells originate from epithelial cells, and in these cases, the epithelial cell type can be selected as the target cell type. Selecting the one or more target cell types in this manner can improve the prediction accuracy of the machine learning model, e.g., by restricting the gene expression data processed by the machine learning model to those cell types that are most relevant to the cancer of interest while excluding cell types that are present in the tumor microenvironment but that are not tumor cells.

In another example, the target cell types can be selected based on the prevalence of different cell types in the training data using for training the machine learning model. More specifically, the machine learning model can be trained on a set of training examples, where each training example includes gene expression data for a training collection of cells. The training examples may be collected and curated such that, for at least a threshold fraction of the training examples (e.g., at least 50%, or at least 80%, or at least 90%, or at least 99% of the training examples), the gene expression data included in the training input is obtained from cells in one or more particular cell types. For instance, most or all of the training examples may include gene expression data derived from cell lines of one or more particular cell types. The system can designate the target cell types as being one or more cell types such that, for at least a threshold fraction of the training examples, the gene expression data included in the training example is derived from cells of the one or more target cell types. Selecting the one or more target cell types in this manner can improve the prediction accuracy of the machine learning model, e.g., by increasing the likelihood that the model inputs processed by the machine learning model at inference are drawn from the same distribution as the model inputs used for training the machine learning model, i.e., thus reducing any distribution shift between training and inference.

The target cell types can be automatically determined by the system, or can be provided to the system by a user, e.g., by way of a graphical user interface or an application programming interface (API) made available by the system.

The system processes the bulk gene expression data to generate cell type-specific gene expression data for each cell type in the set of possible cell types (304). The cell type-specific gene expression data for a cell type defines, for each gene in the set of genes, a predicted expression level of the gene across only cells of the cell type in the collection of cells. That is, the cell type-specific gene expression data deconvolves (disentangles) the bulk gene expression data, which characterizes gene expression over the entire collection of cells, into cell type-specific gene expression data that characterizes respective gene expression data over each possible cell type in the collection of cells.

The system can process the bulk gene expression data to generate the cell type-specific gene expression data in any of a variety of possible ways. For instance, the system can generate the cell type-specific gene expression data using the BayesPrism approach described in Chu et al., “Cell type and gene expression deconvolution with BayesPrism enables Bayesian integrative analysis across bulk and single-cell RNA sequencing in oncology,” Nature Cancer, 2022. As another example, the system can generate the cell type-specific gene expression data using the CIBERSORTx approach described in Im et al., “A Comprehensive Overview of RNA Deconvolution Methods and Their Application,” Mol Cells. 2023 Feb. 28; 46(2): 99-105.

The system extracts the cell type-specific gene expression data for the target cell types, e.g., from an array holding the cell type-specific gene expression data for all the cell types in the set of possible cell types (306). In implementations where the set of target cell types includes more than one cell type, the system can aggregate (e.g., sum) the cell type-specific gene expression data for each target cell type in order to generate target cell type-specific gene expression data that defines, for each gene in the set of genes, a predicted expression level of the gene across only cells of the target cell type(s) in the collection of cells.

The system obtains data identifying a proper subset of the set of genes as being “pathway genes” that participate in a biochemical pathway associated with the target gene (308).

The number of genes in the set of pathway genes can be significantly less than the total number of genes in the set of genes. For instance, the number of genes in the set of pathway genes can be less than 50%, or less than 10%, or less than 1% of the number of genes in the set of genes.

The system reduces the dimensionality of the cell type-specific gene expression data by removing those dimensions that define expression levels of genes that are not included in the set of pathway genes (310). That is, the system maintains only those dimensions of the cell type-specific gene expression data that define gene expression levels for genes that are included in the set of pathway genes, while removing any other dimensions of the cell type-specific gene expression data.

Reducing the dimensionality of the gene expression data can reduce consumption of computational resources (e.g., memory and computing power) by reducing the number of arithmetic operations performed by the machine learning model to process the reduced-dimensionality gene expression data. Further, reducing the dimensionality of the gene expression data can increase the prediction accuracy of the machine learning model, e.g., by reducing the likelihood of the machine learning model overfitting during training of the machine learning model.

In some cases, the system performs steps 302-306, i.e., to transform the bulk gene expression data to cell type-specific gene expression data, without performing steps 308-310, i.e., to reduce the dimensionality of the gene expression data to capture only the pathway genes. In other cases, the system performs steps 308-310, i.e., to reduce the dimensionality of the gene expression data to capture only the pathway genes, without performing steps 302-306, i.e., to transform the bulk gene expression data to cell type-specific gene expression data.

The system outputs the pre-processed gene expression data, e.g., for processing by the machine learning model to generate a predicted gene essentiality score, as described with reference to FIG. 2.

FIG. 4 is a flow diagram of an example process 400 for training a machine learning model to generate a predicted gene essentiality score for a target gene. For convenience, the process 400 will be described as being performed by a system of one or more computers located in one or more locations. For example, a prediction system, e.g., the prediction system 100 of FIG. 1, appropriately programmed in accordance with this specification, can perform the process 400.

The system obtains a set of training examples for training the machine learning model (402). Each training example includes: (i) a training input that includes gene expression data for a training collection of cells, and (ii) a target gene essentiality score for the target gene.

The target gene essentiality scores specified by the training examples can be generated using appropriate experimental techniques, e.g., using gene knockout of the target gene (e.g., using CRISPR-Cas9 to disable the target gene), or using RNA interference (e.g., using small interfering RNA or short hairpin RNA to reduce the expression of the target gene), or using chemical genetic techniques (e.g., where small molecules are used to inhibit the function of the protein coded for by the target gene).

For some or all of the training examples, the training collection of cells characterized by the training input of the training example may correspond to a respective cell line, e.g., a respective cancer cell line.

The system trains the machine learning model on the set of training examples by a machine learning training technique (404). More specifically, the system trains the machine learning model to optimize an objective function that, for each training example, measures a discrepancy between: (i) the target gene essentiality score specified by the training example, and (ii) a predicted gene essentiality score generated by the machine learning model by processing the training input of the training example. The objective function can measure the discrepancy between the target gene essentiality score and the predicted gene essentiality score, e.g., using a squared error metric, or a cross-entropy metric, or any other appropriate metric.

The system can train the machine learning model using any training technique appropriate for the architecture of the machine learning model. For instance, for a machine learning model implemented as a neural network, the system can train the machine learning model using a stochastic gradient descent training technique.

Optionally, for a machine learning model that is configured to generate a predicted gene essentiality score that defines a likelihood that the target gene is an essential gene, the system can determine a threshold for evaluating whether the target gene should be classified as an essential gene or a non-essential gene (406). For instance, if the predicted gene essentiality score is above the threshold, then the system can classify the target gene as being an essential gene; otherwise, the system can classify the target gene as being a non-essential gene. The system can also use the threshold as part of generating a prediction characterizing a patient based on the predicted gene essentiality score for the target gene for the patient, as described above with reference to FIG. 2.

To determine the threshold, the system can determine a respective quality score for each candidate threshold in a set of candidate thresholds. The set of candidate thresholds can include any appropriate number of candidate thresholds, e.g., at least 100 candidate thresholds, and can be evenly spaced over the range of continuous range of possible gene essentiality scores, e.g., the range [0,1]. The system can determine a respective quality score for each candidate threshold, e.g., by determining a precision of the machine learning model, or a recall of the machine learning model, or both, when the machine learning model classifies the training examples of the training data (or a held out validation set) in accordance with the candidate threshold. The system can generate the quality score for each candidate threshold, e.g., as a F1-score that is a harmonic mean of the precision and recall for the candidate threshold. The system can select the candidate threshold associated with the highest quality score as the final threshold.

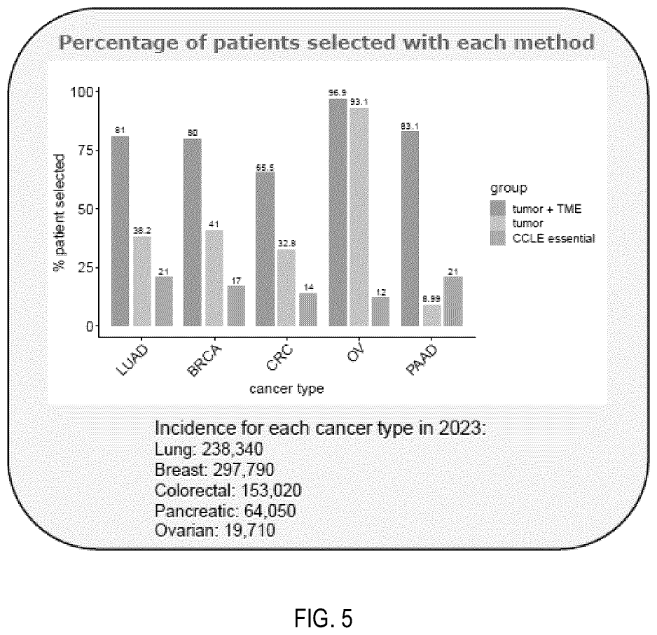

FIG. 5 shows a plot that illustrates an example of the effects of transforming the bulk gene expression data to cell type-specific gene expression data prior to processing the gene expression data using the machine learning model to predict the gene essentiality score for the target gene. More specifically, the horizontal axis on the plot differentiates between types of cancer (e.g., lung, breast, colorectal, pancreatic, and ovarian cancer). The vertical axis reflects the percentage of patients (e.g., in a test set) that are selected by the system, e.g., to receive a drug that targets the target gene, e.g., based on the predicted gene essentiality score for the target gene satisfying a threshold. For each cancer type, the plot shows the percentage of patients selected by the system when: (i) the machine learning model bulk gene expression data (“tumor+TME”), (ii) the machine learning model processes cell type-specific gene expression data (“tumor”), and (iii) the machine learning model processes cell-type specific gene expression data generated by single cell analysis of cancer cell lines (“CCLE essential”). It will be appreciated that, in most cases, transforming the bulk gene expression data to cell type-specific gene expression data significantly reduces the percentage of patients that are selected (e.g., to receive the drug) as compared to processing bulk gene expression data. These results indicate that transforming the bulk gene expression data to cell type-specific gene expression data can cause the machine learning model to become more selective (and potentially more accurate) in predicting gene essentiality.

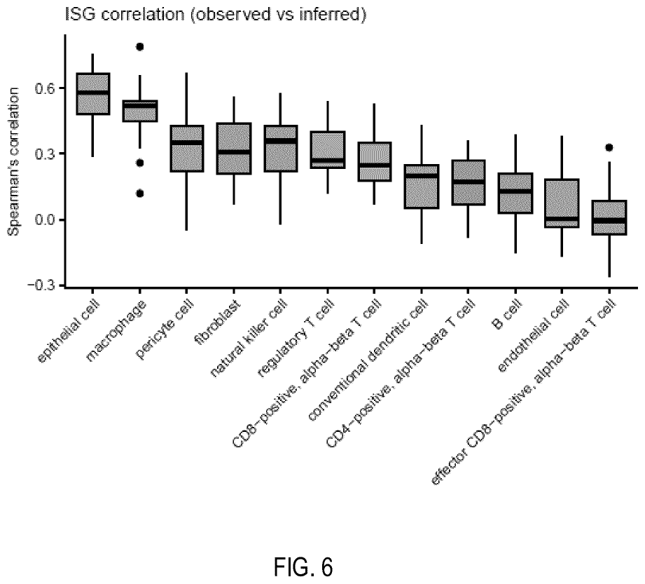

FIG. 6 shows a plot that illustrates, for each cell type in a set of cell types (as shown on the horizontal axis of the plot), a Spearman's correlation (as shown on the vertical axis of the plot) between: (i) predicted cell type-specific gene expression levels for the cell type, and (ii) ground truth cell type-specific gene expression levels for the cell type. The predicted cell type-specific gene expression levels are generated by deconvolving the bulk gene expression data, e.g., as described with reference to FIG. 3. The ground truth cell-type specific gene expression levels are generated by experimental methods. It will be appreciated that, for several cell types (including epithelial cells), the predicted cell-type specific gene expression levels show reasonable correlation with the ground truth cell type-specific gene expression levels. These results indicate that, for at least some cell types, the predicted cell-type specific gene expression levels are reasonably accurate and are appropriate for use in predicting gene essentiality scores.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, or a Jax framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method performed by one or more computers, the method comprising:
obtaining gene expression data for a collection of cells from a patient;
processing a model input comprising the gene expression data using a machine learning model, in accordance with values of a set of machine learning model parameters, to generate a predicted gene essentiality score for a target gene; and
providing the gene essentiality score for the target gene.

2. The method of claim 1, wherein the predicted gene essentiality score for the target gene characterizes whether the target gene is essential for viability of cells in the collection of cells from the patient.

3. The method of claim 1, wherein the gene expression data comprises bulk gene expression data that defines, for each gene in a set of genes, an expression level of the gene across the collection of cells from the patient.

4. The method of claim 1, wherein the gene expression data comprises cell type-specific gene expression data for each of one or more target cell types; and
wherein for each target cell type, the cell type-specific gene expression data for the target cell type defines, for each gene in a set of genes, a predicted expression level of the gene across cells of the target cell type in the collection of cells from the patient.

5. The method of claim 4, further comprising generating the cell type-specific gene expression data by operations including:
obtaining bulk gene expression data that defines, for each gene in the set of genes, an expression level of the gene across the collection of cells from the patient;
processing the bulk gene expression data to generate cell type-specific gene expression data for each cell type in a set of possible cell types, wherein each of the one or more target cell types are included in the set of possible cell types; and
extracting the cell type-specific gene expression data for each of the one or more target cell types from the cell type-specific gene expression data for the set of possible cell types.

6. The method of claim 1, wherein the machine learning model has been trained on a set of training examples by a machine learning training technique.

7. The method of claim 6, wherein each training example in the set of training examples includes: (i) a training input to the machine learning model that comprises gene expression data for a training collection of cells, and (ii) a target gene essentiality score for the target gene.

8. The method of claim 7, wherein training the machine learning model on the set of training examples comprises, for each training example:
training the machine learning model to optimize an objective function that measures a discrepancy between: (i) the target gene essentiality score specified by the training example, and (ii) a predicted gene essentiality score generated by processing the training input of the training example using the machine learning model.

9. The method of claim 7, wherein for one or more of the training examples, the training example is derived from a cell line, and the training input of the training example comprises gene expression data for cells in the cell line.

10. The method of claim 1, wherein the machine learning model comprises one or more of: a neural network model, or a support vector machine learning, or a random forest model, or a linear regression model.

11. The method of claim 1, wherein the collection of cells from the patient are obtained from a biopsy of the patient.

12. The method of claim 11, wherein the biopsy comprises one or more of: a needle biopsy, or a punch biopsy, or an incisional biopsy, or an excisional biopsy, or an endoscopic biopsy, or a bone marrow biopsy, or a blood biopsy.

13. The method of claim 1, wherein the collection of cells are obtained from a biopsy of a cancerous tumor of the patient.

14. The method of claim 13, wherein the cancerous tumor of the patient is a kidney cancer tumor, or a bone cancer tumor, or a lung cancer tumor, or a bladder cancer tumor, or an ovarian cancer tumor, or a colon colorectal cancer tumor, or an endometrial uterine cancer tumor, or a breast cancer tumor, of a pancreatic cancer tumor, or a lymphoma cancer tumor, or a brain cancer tumor, or a bile duct cancer tumor, or a neuroblastoma cancer tumor, or a sarcoma cancer tumor, or a skin cancer tumor, or a gastric cancer tumor, or a head and neck cancer tumor, or an esophageal cancer tumor.

15. The method of claim 1, wherein providing the predicted gene essentiality score for the target gene comprises:
determining whether the patient should receive a drug that target the target gene based at least in part on the predicted gene essentiality score.

16. The method of claim 15, wherein determining whether the patient should receive a drug that target that target gene based at least in part on the predicted gene essentiality score comprises:
determining whether the patient should receive the drug that targets the target gene based at least in part on whether the predicted gene essentiality score satisfies a threshold.

17. The method of claim 15, further comprising, in response to determining that the patient should receive the drug, causing the drug to be administered to the patient.

18. The method of claim 1, wherein providing the predicted gene essentiality score for the target gene comprises:
determining a likelihood that the patient will respond to a drug that targets the target gene based at least in part on the predicted gene essentiality score.

19. A system comprising:
one or more computers; and
one or more storage devices communicatively coupled to the one or more computers, wherein the one or more storage devices store instructions that, when executed by the one or more computers, cause the one or more computers to perform operations comprising:
obtaining gene expression data for a collection of cells from a patient;
processing a model input comprising the gene expression data using a machine learning model, in accordance with values of a set of machine learning model parameters, to generate a predicted gene essentiality score for a target gene; and
providing the gene essentiality score for the target gene.

20. One or more non-transitory computer storage media storing instructions that when executed by one or more computers cause the one or more computers to perform operations comprising:
obtaining gene expression data for a collection of cells from a patient;
processing a model input comprising the gene expression data using a machine learning model, in accordance with values of a set of machine learning model parameters, to generate a predicted gene essentiality score for a target gene; and
providing the gene essentiality score for the target gene.
